(19) United States
(12) Patent Application Publication
(10) Pub. No.: US 2026/0158216 A1
(43) Pub. Date: Jun. 11, 2026

(54) HANDHELD DEVICE WITH ACTIVATION SWITCH FOR PATIENT-CONTROLLED ANALGESIA

(71) Applicant: B. Braun Melsungen AG, Melsungen (DE)

(72) Inventors: Jan Schwarz, Melsungen (DE); Maria Buerger, Malsfeld (DE); Peter Barkowski, Melsungen (DE)

(21) Appl. No.: 19/412,166

(22) Filed: Dec. 8, 2025

(30) Foreign Application Priority Data
Dec. 10, 2024 (EP) 24218772.2

(51) Int. Cl. A61M 5/168; A61M 5/14

(52) U.S. Cl. CPC A61M 5/16877; A61M 2005/1405; A61M 2205/584; A61M 2205/586; A61M 2205/587

(57) ABSTRACT

A handheld device for patient-controlled analgesia includes a handle configured to be gripped and held by a patient with one hand. The handle encloses or defines a volume with a center of volume. The handle includes a first handle section configured to be gripped by a thumb and an index finger or a middle finger of the patient in such a way that an activation switch provided on a first large surface of the handle can be actuated by the patient’s thumb while the patient’s index finger or middle finger can be brought into contact with a defined finger recess provided on a second large surface of the handle. The handle has a center of mass which is offset relative to the center of volume of the handle in a direction away from the first handle section and is located in a second handle section of the handle.

### CROSS-REFERENCE TO RELATED APPLICATIONS

This application claims priority under 35 U.S.C. § 119 to European Application No. 24218772.2, filed on December 10, 2024, the content of which is incorporated by reference herein in its entirety.

### FIELD

The present disclosure relates to a handheld device (control unit) with an activation switch (push button) for patient-controlled analgesia (PCA).

### BACKGROUND

Patient-controlled analgesia, or PCA for short, is a method of administering analgesics to the patient themselves via a parenteral or epidural route using an electronically controlled infusion pump. At the touch of a button, patients can request a pre-programmed dose of pain medication and administer it themselves.

In most cases, painkillers (usually highly potent analgesics from the opiate group) are administered via an intravenous line connected to an infusion pump. The catheter is connected directly to the existing central venous catheter (CVC) or peripheral venous indwelling cannula via a Y-piece.

With this pain therapy, the patient can administer a so-called bolus at the touch of a button. The attending physician determines the dose to be administered. A lock interval prevents another bolus from being injected in quick succession. This prevents accidental or intentional overdose. The patient can administer their pain medication as needed and, for example, pause when they have little or no pain.

This form of pain therapy has been established as the global standard in the post-operative phase since the 1980s. It is also frequently used in chronic pain syndrome and palliative care.

Various types of handheld devices (control units) are known from the prior art, which the patient holds in their hand in order to press the control switch arranged thereon.

WO 2021/064415 A1 discloses a handheld device with a handle for gripping by one hand of the patient and with a switch for operation by the patient’s thumb. The handle has indentations for individual fingers. At an (upper) end section remote from the signal line connection, the switch is arranged in an inclined position. The switch is recessed in the handle.

US 2013/0123745 A1 discloses a handheld device with a handle and a switch button. The handle has a recess for a finger. At an (upper) end section remote from the signal line connection, the switch button is arranged in an inclined position. The switch button protrudes or is raised above the handle. Lighting that serves as a location light is also suggested.

Based on our in-house state of the art, a handheld device with a handle that is box-shaped in the broadest sense and with a switch button is known. More precisely, the handle has two large surfaces that are approximately parallel and opposite each other. The signal line connection is located at the bottom of the handle thus formed. On one of the two large surfaces, the switch button is integrated into the affected large surface.

However, such state-of-the-art handheld devices have proven to be suboptimal in terms of ergonomics and handling for patients.

### SUMMARY

The purpose of this disclosure is to provide a handheld device with improved ergonomics and handling.

The handheld device (control unit, PCA button) according to the disclosure is designed and configured for patient-controlled analgesia. It has a handle that is designed and constructed to be grasped and held by a patient with one hand, and which encloses or defines a volume with a center of volume. The handle has a first (upper) handle section, which is designed and constructed to be gripped by the patient’s thumb on one side and by the patient’s index finger or middle finger on the other side in such a way that an activation switch provided on a first large surface of the handle can be actuated by the patient’s thumb, while the patient’s index finger or middle finger can be brought into contact with a defined finger recess provided on a second large surface of the handle. Accordingly, the activation switch (button) provided on the first large surface of the handle can be operated by the patient’s thumb. The patient’s index finger or middle finger can be placed in the defined finger recess provided on the second large surface of the handle. The two large surfaces should preferably be opposite each other. The handle has a center of mass that is offset in one direction away from the first handle section relative to the center of volume of the handle and is located in a second (lower) handle section of the handle. This shifts the center of mass of the handheld device toward the lower edge of the patient’s hand compared to the state of the art, causing the handheld device to fall into the patient’s hand.

Preferably, the handle of the handheld device has the first handle section for at least the thumb and index finger or middle finger, and the second handle section for at least the ring finger.

The first handle section may have a larger cross-sectional area than the second handle section. This means that the first handle section is preferably thicker and/or larger, in particular wider than the second handle section. The cross-section of the two handle sections is preferably not constant and uniform; in particular, when viewed along its longitudinal axis, the cross-section of the handle preferably increases steadily from the second handle section to the first handle section. The handle has a first large surface and a second large surface opposite to it, both of which preferably extend from the first handle section to the second handle section, or in other words, are each part or section of both the first handle section and the second handle section. The activation switch is located on the first handle section of the first large surface. According to the disclosure, a center of mass of the handle, including the activation switch, which is part of the handle, but without taking into account any signal line that is not part of the handle, is located in the second handle section of the handle.

The term “large surface” should preferably be understood to mean that its length and width are (significantly) greater than the depth of the handle.

Preferably, the two large surfaces are trapezoidal or triangular in shape or have a trapezoidal or triangular shape such that the handle tapers from the first handle section to the second handle section. This trapezoidal or triangular shape of the handle has proven to be highly ergonomic for patients.

If a weight body is placed in the second handle section, the center of mass of the handheld device can be shifted particularly far toward the lower edge of the patient’s hand, allowing the handheld device to fall into the patient’s hand particularly quickly and precisely.

From a manufacturing point of view, it is preferable if the weight body is formed from a casting compound, for example resin, preferably epoxy resin, which is injected or poured into the second handle section through an opening in the handle.

The handle can be designed as a housing. If the housing is closed on all sides (e.g., waterproof), a ventilation opening in the handle is particularly preferred so that air can escape from the housing when the casting compound is injected or poured in.

In most cases, a signal line is connected to the second handle section of the handle, which connects the handle to an infusion pump. The signal line should preferably be reinforced with at least one strand of para-aramid (Kevlar) attached to it on the inside of the handle. This ensures that the handle is particularly resistant to tearing away from the signal cable.

In particular, if the signal line has a certain degree of elasticity or rigidity, it is particularly preferable for the signal line to be connected to the second handle section. Then, the shift of the center of mass to the second handle section, as shown in the disclosure, is advantageous because this section is then also pressed or falls against an elastic force of the signal line into the hand, thus coming into contact with the palm and making it easy to grip.

When the two large surfaces of the handle are roughly parallel to each other, the handheld device offers optimum ergonomics for both left-handed and right-handed users.

Preferably, each of the two large surfaces has three or four edges. Two edges extend from the first to the second handle section, with the two edges of each large surface in the first, preferably wider, handle section being further apart than in the second, preferably narrower, handle section.

If the handle is further developed as a housing, it is particularly preferred if the two large surfaces are formed or attached to respective half-shells of the housing, each of which has a surrounding shell edge. The half-shells can be the same size.

The defined finger recess is particularly preferred as a channel-shaped or trough-shaped depression formed in the handle/first handle section, in particular in the second half-shell of the handle, which extends continuously between the two edges of the second large surface. This allows the handheld device to find a clearly defined contact position (contact height) along the length of the handle on the patient’s index or middle finger, regardless of how much longer the finger is than the depression.

Preferably, the depression increases in size from a point of minimal size toward both edges. The “size” of the depression refers to its depth and/or width. This allows the handheld device to find a particularly well-defined contact position between two angled joints of the index finger.

If the point of minimal size is located in the middle between the two edges of the second large surface, then the overall ergonomic optimum of the handheld device is achieved for both left-handed and right-handed users.

The point of minimal size opposite the activation switch is particularly preferred. This interaction between the angle of the index finger and the thumb effectively forms a swivel bearing for the handheld device, whose axis of rotation extends through the point of minimal size and the activation switch. This allows the center of mass, which is spaced apart from this point according to the disclosure, to cause a defined swiveling of the handle. This allows the handle, especially the second handle section, to fit comfortably and ergonomically into the patient’s palm.

If the activation switch is positioned centrally between the two edges of the first large surface, then the overall ergonomics of the handheld device are optimized for both left-handed and right-handed users.

If the activation switch is a membrane switch, the handheld device can be made waterproof. In particular, the handle is formed with the half-shells and the half-shell edges, which then fit tightly together. This allows the housing to be closed on all sides.

The second large surface can be formed in one piece on the second half-shell, and the first large surface can be an extra component (decorative film) that is attached to the first half-shell in a sealing manner.

The activation switch should preferably be raised above the first large surface and protrude outward. This improves the visibility of the activation switch for patients in the dark and/or without looking.

The activation switch should preferably have a signal color that differs from a color of the first large surface (high contrast). For example, an orange activation switch can be provided on a white first large surface. This improves the visibility of the activation switch for the patient.

Preferably, white (LED) lighting designed as a location light should be provided on the first large surface or on the activation switch. This improves the visibility of the activation switch for patients in the dark.

In a preferred embodiment, the first large surface or the activation switch is provided with (LED) lighting designed as an enabling light, e.g., green. This signals when or if enough time has elapsed for the patient to administer the next bolus.

Preferably, this enabling light should be optionally switchable.

In a preferred embodiment, the handle has a height of 90 to 110 mm. This means that if the index finger is in the depression, the handle does not extend out of an average hand on the side of the little finger, so that the little finger can serve as a support for the second end face (on the second handle section).

### BRIEF DESCRIPTION OF THE DRAWINGS

FIG. 1 shows a handheld device according to the exemplary embodiment of the present disclosure with one hand in a view;

FIG. 2 shows the handheld device from FIG. 1 in another view;

FIG. 2a shows a cross-section of the handheld device from FIG. 2;

FIG. 3 shows the handheld device from the previous Figures in another view; and

FIG. 4 shows the handheld device without a first large surface in a view according to FIG. 1.

### DETAILED DESCRIPTION

An exemplary embodiment of the present disclosure is described below, based on the associated Figures.

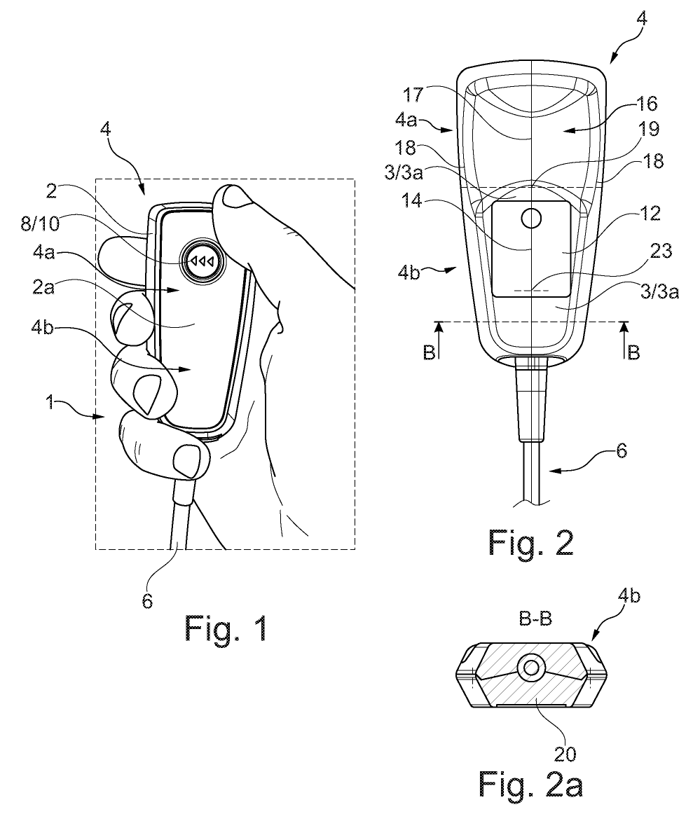

FIG. 1 shows a handheld device 1 according to the exemplary embodiment of the present disclosure with a patient’s hand. The Figure shows a view of a first large surface 2a attached to a first half-shell 2 of a housing-like handle 4. The handle 4 has a length (vertical in FIG. 1) of only about 90–110 mm, so that it is preferably gripped on the rear side only by the patient’s index finger, middle finger, and ring finger. The handle 4 therefore does not extend to the patient’s little finger, so that the little finger preferably supports the handle 4 downwards in the direction of gravity, thereby gripping a connection of a signal line 6. The handle 4 is electrically connected via signal line 6 to an infusion pump (not shown) for pain medication.

The handle 4 can be divided into an upper first handle section 4a shown in FIG. 1 and a lower second handle section 4b shown in FIG. 1. The first handle section 4a is wider than the second handle section 4b. On the second section of the handle 4b, more precisely on a second front side of the handle 4b (in FIG. 1 below), the connection for the signal line 6 is formed, which can be gripped by the little finger.

The first half-shell 2 of the housing-like handle 4 is covered by the essentially flat first large surface 2a, which is designed as an additional component and is attached to the first half-shell 2. However, the first large surface 2a can alternatively also be an (integral) section or component of the first half-shell 2. An activation switch 8 designed as a membrane switch is integrated into the first large surface 2a. Pressing the activation switch 8 with the patient’s thumb causes a bolus of painkiller to be administered via the infusion pump (not shown) connected to the signal line 6.

To ensure visibility in daylight or when the room is lit, the activation switch 8 must be in a contrasting color that stands out from the rest of the first large surface 2a. In the exemplary embodiment shown, the activation switch 8 is colored orange, while the rest of the first large surface 2a is white.

For visibility in the dark, activation switch 8 has LED lighting 10 that serves as a location light.

An LED light in the first large surface 2a serves as an enabling light to indicate that the next bolus can be administered. In the exemplary embodiment shown, the enabling light is green and can be switched on optionally.

FIG. 2 shows the handheld device 1 from FIG. 1 in another view on a second, essentially flat large surface 3a on the rear side with a label 12. In the region of the first handle section 4a, an indentation or depression 16 is provided on the second large surface 3a in a transverse direction to a longitudinal axis 14, which is designed to rest on a finger. This depression 16 has a double cone structure (hyperboloid) that is mirror-symmetrical to the longitudinal axis 14 and aligns itself with a finger of the user, normally the index finger. More precisely, depression 16 has a central point 17 of minimal size at which the width (shown in FIG. 2) and, beyond that, the depth of depression 16 is minimal. From there, the two cones of depression 16 extend with increasing width and depth to the respective edge 18 of the large surface 3a. This alignment geometry, in conjunction with a center of mass 23 shifted into the second handle section 4b, causes the handle 4 to align itself in the hand and allows the activation switch 8 to be pressed using the thumb.

The handle 4, including the activation switch 8, forms a (volumetric) housing that has a center of volume 19. Since the first section 4a of the handle 4, which is furthest away from the connection of the signal line 6, is wider than the second section 4b, the center of volume 19 is further away from the connection of the signal line 6 or the lower edge than from the upper edge. In the exemplary embodiment shown, the center of volume 19 defines a dividing plane perpendicular to the longitudinal axis 14, which forms the boundary between the two sections 4a, 4b. The dividing plane can also be arranged perpendicular to the two large surfaces 2a, 3a and/or, in the case of the approximately trapezoidal handle 4 shown, parallel to the upper and lower (short) edges. The handle 4 has a center of mass 23 which, relative to the center of volume 19 of the handle 4, is displaced in a direction away from the first handle section 4a and is located in a second handle section 4b of the handle 4.

FIG. 2a shows a cross-section of the second handle section 4b along line B-B from FIG. 2. More precisely, the cutting plane B-B runs through a cast-in weight body 20, which causes the center of mass 23 to shift along the longitudinal axis 14 toward the signal line 6, as disclosed.

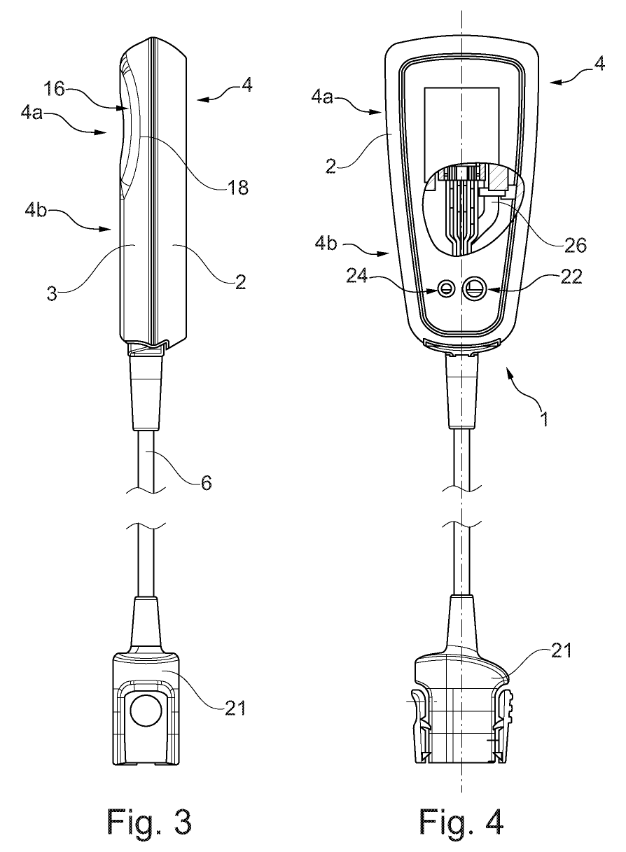

FIG. 3 shows handheld device 1 from the previous Figures in a side view. It can be seen that the two large surfaces 2a and 3a are parallel to each other, creating a box- or shaft-shaped housing from which the handle 4 is formed. The two half-shells 2, 3 each have a circumferential shell edge with which the two half-shells 2, 3 fit together completely tightly. FIG. 3 also shows depression 16 and its rounded transition to the edge 18 shown.

Signal line 6 can be electrically connected to the infusion pump (not shown) via a plug contact 21.

FIG. 4 shows the handheld device 1 in a view of the first half-shell 2 of the handle 4, to which the first substantially flat large surface 2a (shown in FIG. 1) is not yet attached. It can be seen that in the first half-shell 2, in the region of the second handle section 4b, an opening 22 is formed through which the casting compound can penetrate into the inside of the second handle section 4b to form the weight body 20. Furthermore, a ventilation opening 24 is shown through which the displaced air from the waterproof handle 4 can escape.

The signal line 6 is reinforced with at least one strip or strand 26 made of para-aramid (Kevlar), which is attached to its half-shells 2, 3 on the inside the handle 4. This ensures that the handle 4 has a particularly high tear-off resistance relative to the signal cable. Due to a break in the first housing shell 2, a short section of this strand 26 is visible.

A handheld device 1 is disclosed with an activation switch 8, which can also be referred to as a button. The handheld device 1 has a housing-like handle 4 with two large parallel surfaces 2a, 3a, which are formed on or attached to respective half-shells 2, 3 of the handle 4. The edge 18 of each large surface 2, 3 and each half-shell 2, 3, and thus the shape of the entire handle 4, is trapezoidal in the broadest sense, with slightly curved edges 18 and rounded corners. This allows the handle 4 to be divided into a first handle section 4a with a larger cross-section and a second handle section 4b with a smaller cross-section. The first handle section 4a is designed for the patient’s thumb and index finger or middle finger. In the second handle section 4b, a weight or mass 20 is arranged so that this handle section 4b, which is removed from the thumb and index finger, always falls into the patient’s hand and/or swings.

### REFERENCE LIST:

1 handheld device

2 first-half shell

2a first large surface

3 second half-shell

3a second large surface

4 handle

4a first handle section

4b second handle section

6 signal line

8 activation switch

10 location light

12 label

14 longitudinal axis

16 finger recess/depression

17 point of minimal size

18 edge

19 center of volume

20 weight body

21 plug contact

22 opening

23 center of mass

24 ventilation opening

26 strand

1. A handheld device for patient-controlled analgesia, the handheld device comprising a handle configured to be gripped and held by a patient with one hand,
the handle defining a volume with a center of volume,
the handle comprising a first handle section configured to be gripped by a thumb and an index finger or a middle finger of the one hand in such a way that an activation switch provided on a first large surface of the handle is actuatable by the patient’s thumb, while the patient’s index finger or middle finger is positioned to contact a defined finger recess provided on a second large surface of the handle,
the handle further comprising a center of mass that is offset relative to the center of volume in a direction away from the first handle section,
the center of mass located in a second handle section of the handle.

2. The handheld device according to claim 1, further comprising a weight body arranged in the second handle section.

3. The handheld device according to claim 2, wherein the weight body is comprises a casting compound that is injected or poured into the second handle section through an opening arranged in the handle.

4. The handheld device according to claim 3, further comprising a signal line connected to the handle, the signal line being reinforced with at least one strand of para-aramid attached to the signal line on an inside of the handle.

5. The handheld device according to claim 1, wherein each of the first large surface and the second large surface has two edges that extend from the first handle section to the second handle section, wherein the two edges of each of the first large surface and the second large surface are at a greater distance from each other in the first handle section than in the second handle section.

6. The handheld device according to claim 5, wherein the defined finger recess is formed by a channel-shaped or trough-shaped depression in the second large surface, the channel-shaped or trough-shaped depression extending continuously between the two edges of the second large surface.

7. The handheld device according to claim 6, wherein the channel-shaped or trough-shaped depression has an increasing size starting from a point of minimal size toward the two edges of the second large surface.

8. The handheld device according to claim 7, wherein the point of minimal size is arranged centrally between the two edges of the second large surface.

9. The handheld device according to claim 7, wherein the point of minimal size is arranged opposite the activation switch.

10. The handheld device according to claim 1, wherein the activation switch is a membrane switch.

11. The handheld device according to claim 1, wherein the activation switch is raised relative to the first large surface.

12. The handheld device according to claim 1, wherein the activation switch has a signal color that differs from a color of the first large surface.

13. The handheld device according to claim 1, wherein lighting designed as a location light is provided on the first large surface or on the activation switch.

14. The handheld device according to claim 1, wherein lighting designed as an enabling light is provided on the first large surface or on the activation switch.

15. The handheld device according to claim 1, wherein the handle has a length of 90 mm to 110 mm.
